(19) United States
(12) Patent Application Publication
(10) Pub. No.: US 2026/0209687 A1
(43) Pub. Date: Jul. 23, 2026

(54) A STRAIN OF JANIBACTER MELONIS WITH SULFAMETHOXAZOLE DEGRADATION ABILITY AND ITS APPLICATION

(71) Applicant: ZHEJIANG UNIVERSITY OF TECHNOLOGY, Zhejiang (CN)

(72) Inventors: Zhuowei Cheng, Zhejiang (CN); Jianmeng Chen, Zhejiang (CN); Jianming Yu, Zhejiang (CN); Dongzhi Chen, Zhejiang (CN); Chaoyang Lu, Zhejiang (CN); Feifei Cao, Zhejiang (CN)

(21) Appl. No.: 19/417,858

(22) Filed: Dec. 12, 2025

(30) Foreign Application Priority Data
Jan. 21, 2025 (CN) 202510091624.7

(51) Int. Cl. C12N 1/205; B09B 3/60; B09B 101/65; C12R 1/01

(52) U.S. Cl. CPC C12N 1/205; B09B 3/60; B09B 2101/65; C12R 2001/01

(57) ABSTRACT

The present invention discloses a strain of Janibacter melonis with sulfamethoxazole degradation ability and its application. The Janibacter melonis YDW provided by the present invention is isolated from sewage treatment plant sludge, has an efficient degradation effect on sulfamethoxazole, and can degrade the pollutant relatively completely, thus having broad application prospects in the biological purification of industrial wastewater. The Janibacter melonis of the present invention can completely degrade sulfamethoxazole, and the removal rate for 10-20 mg/L sulfamethoxazole under pH 6-7 conditions within 144 h is as high as 97%.

### REFERENCE TO AN ELECTRONIC SEQUENCE LISTING

The content of the electronic sequence listing (C-Sequence_Listing. XML; Size: 3242 bytes; and Date of Creation: Dec. 2, 2025) is herein incorporated by reference in its entirety.

### TECHNICAL FIELD

The present invention relates to a strain of Janibacter melonis with sulfamethoxazole degradation ability and its application.

### BACKGROUND ART

Sulfonamide antibiotics are a class of synthetic antibacterial agents, which are a general term for antibiotic substances containing the p-aminobenzenesulfonamide structure in their molecular structure. Sulfamethoxazole, as a typical sulfonamide antibiotic, has a broad antibacterial spectrum and strong antibacterial effect, particularly effective against Staphylococcus aureus and Escherichia coli, and is commonly used to treat respiratory, urinary, and intestinal infections.

With the widespread use of sulfamethoxazole, and due to its low bioavailability and poor degradability in aquatic environments, it has attracted widespread attention due to its potential toxicity to aquatic animals. Studies have shown that sulfamethoxazole may affect the growth and development of zebrafish. Furthermore, it may cause various negative effects on human health, such as skeletal deformities, liver damage, and gastrointestinal problems. Long-term consumption of water and aquatic products containing sulfamethoxazole may lead to teratogenic and mutagenic risks. Residual sulfamethoxazole in the water environment poses a serious threat to ecological balance, human health, and drinking water safety.

Therefore, researching the efficient degradation of sulfamethoxazole in the environment is of great significance for the ecological environment and human health. Literature searches indicate that there have been no reports on Janibacter melonis regarding this aspect.

### SUMMARY OF THE INVENTION

The object of the present invention is to provide a strain of Janibacter melonis with sulfamethoxazole degradation ability and its application. The Janibacter melonis YDW of the present invention can efficiently degrade sulfamethoxazole using glucose as a co-metabolic substrate, and its growth environment is mild and easy for scale-up cultivation. The discovery of this degrading bacterium has important practical application value for the efficient purification of sulfonamide antibiotic pollutants in pharmaceutical wastewater.

The technical solution adopted by the present invention is:

The present invention provides a new sulfamethoxazole-degrading bacterium—Janibacter melonis YDW, deposited with the China Center for Type Culture Collection (CCTCC) under accession number CCTCC NO: M 20242523, with a deposit date of Nov. 11, 2024, and address: Wuhan University, Wuhan 430072, China.

The basic characteristics of the Janibacter melonis YDW of the present invention are: colonies are yellow, disc-shaped, without spores, without flagella; edges are neat, opaque, easy to pick, bacterial lawn grows along the streak; aerobic, Gram-positive.

The present invention also provides an application of Janibacter melonis YDW in degrading sulfamethoxazole, specifically, the application comprises:
scale-up culturing the Janibacter melonis YDW to obtain a bacterial suspension or centrifuging the bacterial suspension to obtain resting cells, adding the bacterial suspension or resting cells to an inorganic salt culture solution containing glucose and sulfamethoxazole with a pH value of 4.0-9.0, and incubating at 25-35° C. and 100-200 rpm to achieve co-metabolic degradation of sulfamethoxazole and glucose.

Further, in the inorganic salt culture solution, the amount of bacterial suspension or resting cells added, calculated based on dry cell weight, is 20-80 mg/L, preferably 50 mg/L.

Further, the initial concentration of sulfamethoxazole added to the inorganic salt culture solution is 10-175 mg/L, preferably 10-50 mg/L.

Further, the concentration of sterile glucose added to the inorganic salt culture solution is 0.5-5 g/L, preferably 1 g/L.

Further, the composition of the inorganic salt culture solution is: K₂HPO₄ 0.719 g/L, KH₂PO₄ 0.234 g/L, NaNO₃ 1.7 g/L, NH₄Cl 0.98 g/L, MgCl₂ 6H₂O 0.2033 g/L, CaCl₂·2H₂O 0.011 g/L, FeCl₃ 0.0162 g/L, trace element stock solution 10 mL/L, solvent is ultrapure water, pH 7.0; wherein the composition of the trace element stock solution is: CuSO₄·5H₂O 0.02 g/L, FeSO₄·7H₂O 1.0 g/L, MnSO₄·4H₂O 0.1 g/L, NaMoO₄·2H₂O 0.02 g/L, CoCl₂·6H₂O 0.02 g/L, H₃BO₃ 0.014 g/L, ZnSO₄·7H₂O 0.10 g/L, solvent is ultrapure water.

Further, the resting cells of Janibacter melonis YDW are prepared according to the following steps:

### (1) Slant Culture:

inoculating the Janibacter melonis YDW onto a slant LB solid medium, culturing at 30° C. for 24-36 h to obtain slant-grown bacterial cells; wherein the final composition of the LB solid medium is: NaCl 10 g/L, tryptone 10 g/L, yeast extract 5 g/L, agar 18-20 g/L, solvent is ultrapure water, natural pH.

### (2) Scale-Up Culture

picking the slant-grown bacterial cells obtained in step (1) with an inoculation loop and inoculating them into an LB liquid medium, culture at 30° C. and 160 rpm for 24-36 h to obtain a bacterial suspension with OD₆₀₀=0.1-0.2, centrifuging the suspension, collecting wet cells, washing the wet cells with an inorganic salt culture solution to obtain resting cells of Janibacter melonis YDW; the final composition of the LB liquid medium is: NaCl 10 g/L, peptone 10 g/L, yeast extract 5 g/L, solvent is ultrapure water, natural pH.

The Janibacter melonis YDW of the present invention can also be used to degrade sulfonamide organic pollutants.

Compared with the prior art, the beneficial effects of the present invention are reflected in:

The Janibacter melonis YDW provided by the present invention is isolated from sewage treatment plant sludge, has an efficient degradation effect on sulfamethoxazole, and can degrade the pollutant relatively completely, thus having broad application prospects in the biological purification of industrial wastewater.

The Janibacter melonis of the present invention can completely degrade sulfamethoxazole, and the removal rate for 10-20 mg/L sulfamethoxazole under pH 6-7 conditions within 144 h is as high as 97%.

### BRIEF DESCRIPTION OF THE DRAWINGS

FIG. 1: Photograph of the colonial morphology of strain YDW on LB medium.

FIG. 2: Transmission electron micrograph of strain YDW.

FIG. 3: Phylogenetic tree of strain YDW.

FIG. 4: Degradation curve of sulfamethoxazole at different concentrations by Janibacter melonis YDW.

FIG. 5: Degradation curve of 50 mg/L sulfamethoxazole by Janibacter melonis YDW at different pH values.

FIG. 6: Graph of culture OD₆₀₀ versus dry cell weight.

### DETAILED DESCRIPTION OF THE INVENTION

The present invention is further described below in conjunction with specific examples, but the scope of protection of the present invention is not limited thereto:

The materials, reagents, etc. used in the following examples, unless otherwise specified, can be obtained from commercial sources.

The composition of the inorganic salt culture solution is: K₂HPO₄ 0.719 g/L, KH₂PO₄ 0.234 g/L, NaNO₃ 1.7 g/L, NH₄Cl 0.98 g/L, MgCl₂·6H₂O 0.2033 g/L, CaCl₂) 2H₂O 0.011 g/L, FeCl₃ 0.0162 g/L, trace element stock solution 10 ml/L, solvent is ultrapure water, pH 7.0; wherein the composition of the trace element stock solution is: CuSO₄·5H₂O 0.02 g/L, FeSO₄·7H₂O 1.0 g/L, MnSO₄·4H₂O 0.1 g/L, NaMoO₄·2H₂O 0.02 g/L, CoCl₂·6H₂O 0.02 g/L, H₃BO₃ 0.014 g/L, ZnSO₄·7H₂O 0.10 g/L, solvent is ultrapure water.

The final composition of the LB solid medium is: NaCl 10 g/L, tryptone 10 g/L, yeast extract 5 g/L, agar 18-20 g/L, solvent is ultrapure water, natural pH.

The final composition of the LB liquid medium is: NaCl 10 g/L, tryptone 10 g/L, yeast extract 5 g/L, solvent is ultrapure water, natural pH.

### Example 1: Isolation, Purification and Identification of Strain YDW

### 1. Isolation and Purification of Strain YDW.

Strain YDW is a Gram-positive bacterium obtained through acclimation and isolation from activated sludge taken from a sewage treatment plant. The specific steps are as follows:

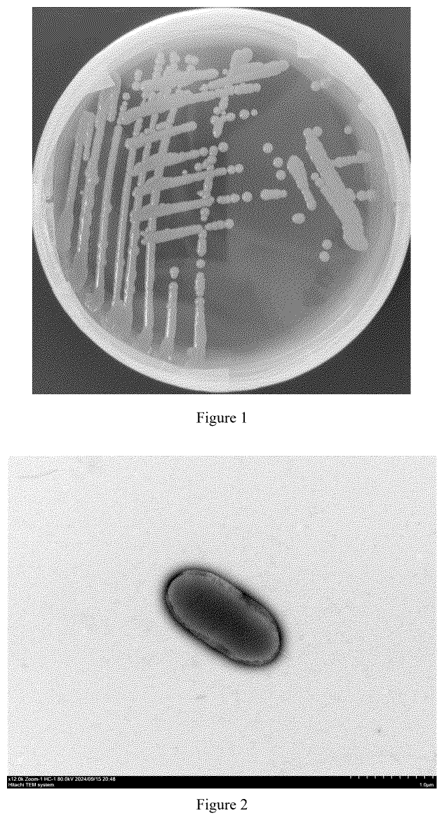

10 mL of activated sludge and 50 mg/L of sulfamethoxazole were added to a 300 mL shake flask containing 100 mL of inorganic salt culture solution for enrichment culture. When the concentration of sulfamethoxazole decreased to 50% of the initial concentration, 5 mL of the enrichment solution was taken and transferred to 100 mL of fresh inorganic salt culture solution, the same concentration of sulfamethoxazole was added, the above enrichment process was repeated 5 times. Then, the last enrichment solution was gradient diluted and spread on LB solid medium, single colonies were selected for isolation and streak plate purification (FIG. 1). The obtained candidate bacteria were then added to the inorganic salt culture solution with sulfamethoxazole for verification, strains with a certain degradation effect were selected, glucose was added as an additional carbon source to enhance degradation, and the target strain YDW was obtained. The target strain YDW's morphology was determined by transmission electron microscopy (FIG. 2).

### 2. Identification of Strain YDW

(1) Characteristics of strain YDW: Colonies are yellow or light yellow, disc-shaped; with neat edges, opaque, and easy to pick. Under transmission electron microscopy, the bacterial cells are elliptical, non-flagellated, and Gram-stain positive.

(2) Identification by 16S rRNA Sequence Analysis and Physiological-Biochemical Experiments.

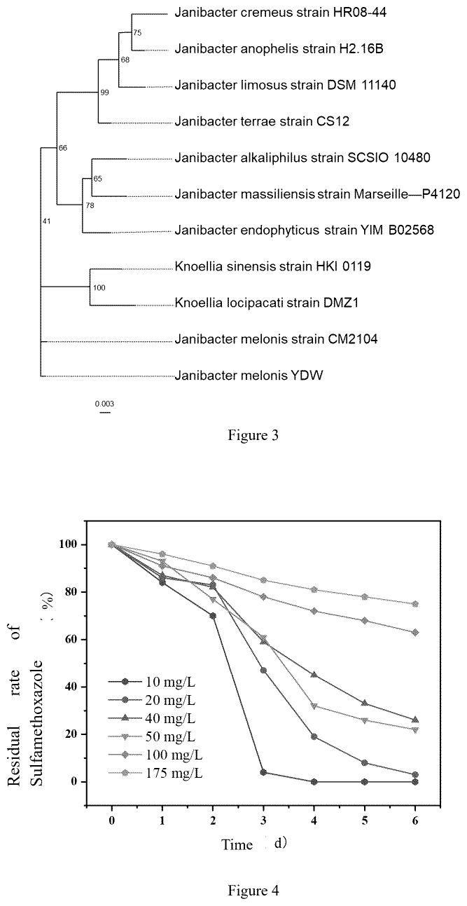

The genomic DNA of strain YDW was extracted and purified by using a nucleic acid reagent [PrepMan Ultra Kits Nucleic Acid Extraction Reagent (ThermoFisher)], and stored at 4° C. The purified DNA was amplified by PCR using universal bacterial primers, forward primer (27F): 5′-AGAGTTTGATCCTGGCTCAG-3′ and reverse primer (1492R): 5′-GGTTACCTTGTTACGACTT-3′. The PCR program was set as follows: pre-denaturation at 94° C. for 4 min; then 30 cycles of denaturation at 94° C. for 45 s, annealing at 55° C. for 45 s, extension at 72° C. for 1 min, and final extension at 72° C. for 10 min. The PCR product was purified and recovered, then sequenced (Zhejiang Tianke High-tech Development Co., Ltd. (formerly Zhejiang Institute of Microbiology)). The 16S rRNA sequencing result was uploaded to NCBI, and the accession number PQ517017 was obtained. Simultaneously, this sequence was aligned with gene sequences in the NCBI database via Blast. It was found to belong to the Janibacter genus, with 99.78% similarity to Janibacter melonis. 10 representative strains were selected from the results, and a phylogenetic tree was constructed using MEGA7.0 in software based on the homology of 16S rRNA gene sequences, as shown in FIG. 3. Based on genetic distance and 16S rRNA sequence alignment, it was identified as Janibacter melonis.

(3) Utilization of 43 Carbon Sources on bioMérieux GP Card by Strain YDW The carbon source metabolism of strain YDW for 43 different carbon sources was investigated using the bioMérieux automatic identification system (entrusted to Zhejiang Tianke High-tech Development Co., Ltd. (formerly Zhejiang Institute of Microbiology)). The identification results are shown in Table 1. According to the bioMérieux VITEK biochemical reaction, strain YDW can strongly utilize 6 carbon sources and cannot utilize the other 37 carbon sources.

TABLE 1
Results of bioMérieux VITEK biochemical
reaction for strain YDW (GP card)
Test Result

| | | | Test |
|---|---|---|---|
| number | abbreviation | English name | result |
| 1 | aMY | D-Amygdalin | − |
| 2 | BgUR | β-Glucuronidase | − |
| 3 | dXYL | D-Xylose | − |
| 4 | aDH1 | Arginine Dihydrolase 1 | + |
| 5 | BgaL | β-Galactosidase | − |
| 6 | agLU | α- Galactosidase | + |
| 7 | aDH2s | Arginine Dihydrolase 2 | − |
| 8 | aMaN | α-Mannosidase | − |
| 9 | PHOS | Phosphatase | − |
| 10 | Leua | Leucine Arylamidase | + |
| 11 | Proa | L-Proline Arylamidase | + |
| 12 | BgURr | β-Glucuronidase | − |
| 13 | agaL | α-Galactosidase | − |
| 14 | Pyra | L-Pyrrolidonyl Arylamidase | − |
| 15 | PIPLc | Phosphatidylinositol Phospholipase C | − |
| 16 | alaa | Alanine Arylamidase | − |
| 17 | aPPa | Alanine-Phenylalanine-Proline | (−) |
| | | Arylamidase | |
| 18 | aspa | L-Aspartate Arylamidase | − |
| 19 | O129R | O/129 Resistance (Comp. Vibrio.) | − |
| 20 | BgaR | β-Galactopyranosidase | − |
| 21 | OPtO | Optochin Resistance | − |
| 22 | URE | UREaSE | − |
| 23 | tyra | Tyrosine Arylamidase | − |
| 24 | dgaL | D-Galactose | − |
| 25 | dRIB | D-Ribose | − |
| 26 | Lac | Lactose | − |
| 27 | dMaL | D-Maltose | − |
| 28 | BacI | Bacitracin Resistance | − |
| 29 | dMaN | D-Mannitol | − |
| 30 | dMNE | D-Mannose | − |
| 31 | PUL | Pullulan | − |
| 32 | dRaF | D-Raffinose | − |
| 33 | SaL | Salicin | − |
| 34 | Sac | Sucrose | + |
| 35 | dtRE | D-Trehalose | + |
| 36 | dSOR | D-Sorbitol | − |
| 37 | URE | UREaSE | − |
| 38 | POLYB | Polymyxin B Resistance | − |
| 39 | cDEX | Cyclodextrin | − |
| 40 | Nag | N-Acetyl-D-Glucosamine | − |
| 41 | ILatK | L-Lactate Alkalinization | − |
| 42 | Nc6.5 | Growth in 6.5% NaCl | − |
| 43 | MBdg | Methyl-B-D-Glucopyranoside | − |

card GP Analysis time 5.00 h
Note,
+: positive reaction;
−: negative reaction

### Example 2: Obtaining Resting Cells of Janibacter melonis YDW

### 1. Slant Culture:

Janibacter melonis YDW was inoculated into LB liquid medium, cultured at 30° C., 160 rpm for 24-36 h, then the activated bacteria was streaked on a solid LB plate and cultured in a 30° C. incubator. A single colony was picked, and streak plating was continued to check the purity of the bacteria, followed by routine storage on an LB agar slant in a test tube at 4° C.

### 2. Scale-Up Culture

The slant-grown cells from step 1 were inoculated into LB liquid medium, cultured at 30° C., 160 rpm for 24-36 h to obtain a bacterial suspension with OD₆₀₀=0.1-0.2. The suspension was centrifuged to collect wet cells, which were subsequently washed with an inorganic salt culture solution to obtain resting cells of Janibacter melonis YDW.

### Example 3: Detection of the Degradation Performance of Janibacter melonis YDW Against Sulfamethoxazole at Different Concentrations

The inorganic salt culture solution was dispensed into 300 mL flasks, 100 mL per flask, sterilized at 110° C. for 40 min. After sterilization, the 300 mL flasks were left at room temperature for 2 days to confirm no contamination. Resting cells obtained by the method of Example 2 were added to a final concentration of 50 mg/L (based on dry cell weight), then 1 g/L glucose and sulfamethoxazole were added as carbon sources to final concentrations of 10, 20, 40, 50, 100, 175 mg/L respectively. The flasks were sealed and cultured in a shaker at 30° C., 160 rpm, and blank controls without bacteria were performed. The concentration of residual sulfamethoxazole was measured in the flasks daily, and the residual rate curve of sulfamethoxazole at different initial concentrations over time for the strain was plotted. The results are shown in FIG. 4. The results show that when the sulfamethoxazole concentration is below 20 mg/L, strain YDW can rapidly degrade all the added substrate.

The concentration of sulfamethoxazole was determined by a Waters e2695 high-performance liquid chromatograph. Liquid chromatography column parameters: Agilent StableBond Analytical (4.6×250 mm), mobile phase: 0.1% acetic acid water:acetonitrile (v:v=40:60), detection wavelength: 264 nm, flow rate: 1.0 mL·min⁻¹, column temperature: 30° C.; injection volume: 20 μL, injection time: 5 min.

### Example 4: Detection of the Degradation Performance of Janibacter melonis YDW Against 50 mg/L Sulfamethoxazole Under Different Initial pH Environments

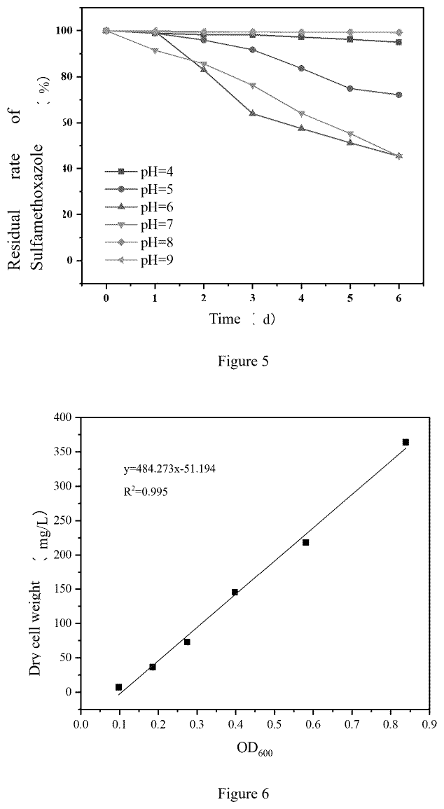

The inorganic salt culture solution was adjusted to different pH values (4.0, 5.0, 6.0, 7.0, 8.0, 9.0) using 1 mol/L NaOH aqueous solution or 1 mol/L HCl aqueous solution. Under the condition of an initial sulfamethoxazole concentration of 50 mg/L, 1 g/L sterile glucose was added, and inoculation was performed with the bacterial suspension prepared by the method of Example 2, so that the initial bacterial concentration in each parallel sample was 50 mg/L based on dry cell weight. The samples were placed in a constant temperature shaker at 30° C., 160 rpm for oscillating culture, and a blank control without bacteria was performed. The concentration of residual sulfamethoxazole in the flasks was measured daily, and the residual rate curve of sulfamethoxazole over time under different pH environments for the strain was plotted. The results are shown in FIG. 5. The results show that Janibacter melonis YDW can effectively degrade sulfamethoxazole under neutral and slightly acidic conditions, and the degradation effect on sulfamethoxazole is best at pH 6.0 and 7.0.

Method for determining dry cell weight and OD₆₀₀: Janibacter melonis YDW was inoculated into sterile medium and cultured until the logarithmic growth phase. A certain volume of the cell culture in the logarithmic growth phase was taken and transferred to a sterile centrifuge tube, centrifuged, and the supernatant was discarded, the cell pellet was resuspended with an appropriate amount of sterile physiological saline to make a cell suspension. An appropriate amount of the prepared cell suspension was transferred for the purpose of measuring its OD₆₀₀ value by using a visible spectrophotometer. The cell suspension was appropriately diluted, and the OD₆₀₀ value at the dilution was measured, the measurement was repeated 3 times for each dilution, and the average value was taken. A certain volume of the above cell suspension was taken and transferred to a pre-weighed sterile centrifuge tube with a mass of m₁, the cell suspension was centrifuged to pellet the cells at the bottom of the tube, the supernatant was carefully discarded, then an appropriate amount of ultrapure water was slowly added to the centrifuge tube, mixed well to disperse the cells, centrifuged again and the supernatant was discarded, the operation was repeated 2-3 times to remove impurities such as residual medium components on the cell surface. Then the centrifuge tube (with the cell pellet) was placed in a drying oven, a suitable temperature of 80° C. was set, drying was conducted until the centrifuge tube and the cell pelleted inside reached constant weight (the difference between two consecutive weighings is within ±0.1 mg), the final mass was recorded as m₂. Dry cell weight (mg/L)=(m₂-m₁)/sampling volume (L). According to this method, the dry cell weight of the cell suspensions corresponding to different OD₆₀₀ values was determined respectively. The measured OD₆₀₀ value (average value) of each diluted cell suspension and the corresponding calculated dry cell weight value were plotted, with the OD₆₀₀ value as the abscissa and the dry cell weight value (mg/L) as the ordinate. The results are shown in FIG. 6.

Although the present invention has been disclosed above in the form of examples, it is not intended to limit the scope of protection of the present invention. Any modifications and alterations made by those skilled in the art without departing from the spirit and scope of the present invention shall fall within the scope of protection of the present invention.

1. A strain of Janibacter melonis YDW with sulfamethoxazole degradation ability, deposited with the China Center for Type Culture Collection (CCTCC) under accession number CCTCC NO: M 20242523, with a deposit date of Nov. 11, 2024, and address: Wuhan University, Wuhan 430072, China.

2. An application of the Janibacter melonis YDW according to claim 1 in degrading sulfamethoxazole.

3. The application according to claim 2, wherein the application comprises: scale-up culturing the Janibacter melonis YDW to obtain a bacterial suspension or centrifuging the bacterial suspension to obtain resting cells, adding the bacterial suspension or resting cells to an inorganic salt culture solution containing glucose and sulfamethoxazole with a pH value of 4.0-9.0, and incubating at 25-35° C. and 100-200 rpm to achieve degradation of sulfamethoxazole.

4. The application according to claim 3, wherein the initial concentration of sulfamethoxazole in the inorganic salt culture solution is 10-175 mg/L.

5. The application according to claim 3, wherein the amount of bacterial suspension or resting cells added to the inorganic salt culture solution, calculated based on dry cell weight, is 20-80 mg/L.

6. The application according to claim 3, wherein the concentration of sterile glucose added to the inorganic salt culture solution is 0.5-5 g/L.

7. The application according to claim 3, wherein the composition of the inorganic salt culture solution is: KHPO₄ 0.719 g/L, KH₂PO₄ 0.234 g/L, NaNO₃ 1.7 g/L, NH₄Cl 0.98 g/L, MgCl₂·6H₂O 0.2033 g/L, CaCl₂): 2H₂O 0.011 g/L, FeCl₃ 0.0162 g/L, trace element stock solution 10 ml/L, solvent is ultrapure water, pH 7.0; wherein the composition of the trace element stock solution is: CuSO₄·5H₂O 0.02 g/L, FeSO₄·7H₂O 1.0 g/L, MnSO₄·4H₂O 0.1 g/L, NaMoO₄·2H₂O 0.02 g/L, CoCl₂·6H₂O 0.02 g/L, H₃BO₃ 0.014 g/L, ZnSO₄·7H₂O 0.10 g/L, solvent is ultrapure water.

8. The application according to claim 3, wherein the resting cells of Janibacter melonis YDW are prepared according to the following steps:
(1) Slant culture: inoculating the Janibacter melonis YDW onto a slant LB solid medium, culturing at 30° C. for 24-36 h to obtain slant-grown bacterial cells;
(2) Scale-up culture: picking the slant-grown bacterial cells obtained in step (1) with an inoculation loop and inoculating them into an LB liquid medium, culturing at 30° C. and 160 rpm for 24-36 h to obtain a bacterial suspension with OD₆₀₀=0.1-0.2, centrifuging the suspension, collecting wet cells, then washing the wet cells with an inorganic salt culture solution to obtain resting cells of Janibacter melonis YDW.


SEQUENCE LISTING (1 sequence)

SEQ ID NO: 1 - DNA, 1381 bp, Janibacter melonis
  source 1..1381: mol_type = genomic DNA
gtcgaacggtgaaccttggagcttgctctaaggggatcagtggcgaacgggtgagtaacacgtgagtaacctgccccggactctggaataagcgctggaaacggcgtctaataccggatacgagacgcaaccgcatggttagcgtctggaaagtttttcggtctgggatggactcgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcaccggctaactacgtgccagcagccgcggtaatacgtagggtgcgagcgttgtccggaattattgggcgtaaagagcttgtaggcggtttgtcgcgtctgctgtgaaaatccggggctcaaccccggacttgcagtgggtacgggcagactagagtgtggtaggggagactggaattcctggtgtagcggtgaaatgcgcagatatcaggaggaacaccgatggcgaaggcaggtctctgggccactactgacgctgagaagcgaaagcatggggagcgaacaggattagataccctggtagtccatgccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacatccagagatgggtgccccttttggtcggtatacaggtggtgcatggttgtcgtcagctcgtgtcgtgagatgttgggttaagtcccgcaacgagcgcaaccctcgttctatgttgccagcacgtcatggtggggactcataggagactgccggggtcaactcggaggaaggtggggatgacgtcaaatcatcatgccccttatgtcttgggcttcacgcatgctacaatggccggtacaaagggctgcgataccgcaaggtggagcgaatcccaaaaaaccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcccaacccttgtgggggg